(19) United States
(12) Patent Application Publication
(10) Pub. No.: US 2012/0226278 A1
(43) Pub. Date: Sep. 6, 2012

(54) Intramedullary Nail

(76) Inventors: Reto NARDINI, Langendorf (CH); Laura VILLIGER, Langendorf (CH); Robert FRIGG, Langendorf (CH)

(21) Appl. No.: 13/222,675

(22) Filed: Aug. 31, 2011

Related U.S. Application Data
(60) Provisional application No. 61/384,548, filed on Sep. 20, 2010.

(51) Int. Cl. A61B 17/58

(52) U.S. Cl. 606/64

(57) ABSTRACT

An intramedullary nail extending from a proximal end to a distal end comprises a distal portion extending longitudinally from the distal end to a first connecting end and including a first locking hole extending therethrough; a proximal end extending longitudinally from a second connecting end to the proximal end of the nail and including a second locking hole extending transversely therethrough and a joint connecting the first and second connecting ends such that the distal and proximal portions are rotatable relative to one another about the joint.

### PRIORITY CLAIM

The present application claims priority to U.S. Provisional Application Ser. No. 61/384,548 filed on Sep. 20, 2010 and entitled “Intramedullary Nail,” the entire disclosure of which is incorporated herein by reference.

### FIELD OF THE INVENTION

The present invention generally relates to an intramedullary nail and, in particular, to an intramedullary nail with an adaptable shape.

### BACKGROUND

Intramedullary nails with adaptable shape are known in the art but none of these known devices offers the possibility of distal and proximal locking. Adaptable intramedullary nails are particularly relevant to applications involving insertion of the intramedullary nail in the humerus. After a fracture of the proximal humerus, the humeral head is often dislocated. When using a conventional intramedullary nailing technique, no open reduction is possible. Once the location of the entry hole for the nail has been defined and drilled, the inserted nail is used to reduce the humeral head fragment. In some cases, the selection of the location of the entry hole may not be optimal, resulting in a suboptimal reduction with no method for corrective action. Although the shoulder joint is less susceptible to suboptimal reduction than other joints, angles over 20° can lead to reduced mobility and pain for the patient.

### SUMMARY OF THE INVENTION

The present invention relates to an intramedullary nail adaptable in shape to correct a misplaced insertion of the nail in the intramedullary cavity while also allowing transverse distal and proximal locking.

According to one exemplary embodiment of the present invention, the intramedullary nail includes distal and proximal portions connected via a joint, both of the distal and proximal portions including transverse locking holes.

Some of the advantages of the intramedullary nail according to the invention are that:

a position of the inserted intramedullary nail may be corrected by moving the proximal and the distal portions of the intramedullary nail relative to one another via the joint;

bone fragments may be fixed relative to one another by inserting proximal and distal transverse locking screws through the intramedullary nail to fix proximal and distal bone fragments relative to one another;

bone portions of, for example, the tibia, femur or humerus, positioned via an osteotomy may be fixed at a predefined angle; and

it is possible to select and drill an entry hole such that the proximal locking screws will not interfere with present facture lines.

In another exemplary embodiment, there are at least 2 transverse locking holes in the proximal and/or distal portions.

A ratio of a length of the distal portion L_{D} to a length of the proximal portion L_{P} is larger than 2, and preferably larger than 3. For example, in a specific case, the ratio L_{D}: L_{P} has been established to be 4.26.

In a further exemplary embodiment, the joint is located at a distance D≦L/3 from a proximal end of the nail, where Lisa total length of the nail, and more preferably at a distance D≦L/4 from the proximal end.

In a further exemplary embodiment, at least one of the distal and proximal portions, and preferably both the distal and proximal portions, is/are free of any outer threads.

The joint includes a releasable locking mechanism. For example, the locking mechanism may include interlocking toothed surfaces arranged on each joint member. In another example, the locking mechanism includes a radially compressible sleeve with a concave spherical cavity receiving a convex spherical male joint member.

In a further exemplary embodiment, the intramedullary nail further comprises a fastening screw inserted through an opening at its proximal end to releasably lock the joint. An advantage of this embodiment is that the joint may be locked at the proximal end of the nail via the entry hole through which the intramedullary nail has been inserted so that no further incision is required. This locking mechanism permits the joint to be locked and unlocked even after placement of the locking screws. Direct locking of the joint would require another surgical approach resulting in a deterioration of the already weakened bone.

The joint can be of any type. In one exemplary embodiment, the joint is a ball-and-socket joint permitting angulation in multiple directions. In another embodiment the joint is a hinge permitting the proximal and distal portions to pivot about the joint relative to one another. A hinge joint may provide higher mechanical stability of the locked joint while lowering manufacturing costs and facilitating distal locking.

In another exemplary embodiment, the nail has more than one joint.

In one exemplary embodiment, the nail is sized and shaped for insertion into the intramedullary cavity of a long bone, e.g. the humerus.

The nail may be formed of any biocompatible material such as, for example, titanium, stainless steel, and cobalt chrome. Cobalt chrome may be used to improve mechanical stability.

### BRIEF DESCRIPTION OF THE DRAWINGS

Several embodiments of the invention will be described in the following by way of example and with reference to the accompanying drawings in which:

FIG. 1 illustrates a lateral view of an embodiment of the intramedullary nail according to the invention;

FIG. 2 illustrates a longitudinal section through the embodiment of the intramedullary nail of FIG. 1;

FIG. 3 illustrates a longitudinal section through another embodiment of the intramedullary nail according to the invention with the proximal and distal part coaxially arranged;

FIG. 4 illustrates a longitudinal section through the embodiment of the intramedullary nail of FIG. 3 with the proximal and distal part in an angled position;

FIG. 5 illustrates a perspective view of a further embodiment of the intramedullary nail according to the invention;

FIG. 6 illustrates a perspective view of the embodiment of the intramedullary nail of FIG. 5 with a proximal part of a humerus;

FIG. 7 illustrates a lateral view of the embodiment of the intramedullary nail of FIG. 5; and

FIG. 8 illustrates a longitudinal section through the proximal part of the embodiment of the intramedullary nail of FIG. 5.

### DETAILED DESCRIPTION

The present invention may be further understood with reference to the following description and the appended drawings, wherein like elements are referred to with the same reference numerals. The present invention relates to the treatment of fractures and, in particular, to treatments using intramedullary nails. Exemplary embodiment of the invention describe an intramedullary nail including a joint permitting proximal and distal portions thereof to rotate relative to one another such that, after insertion of the intramedullary nail, a reduction of the fracture may be adjusted and locked in a desired configuration.

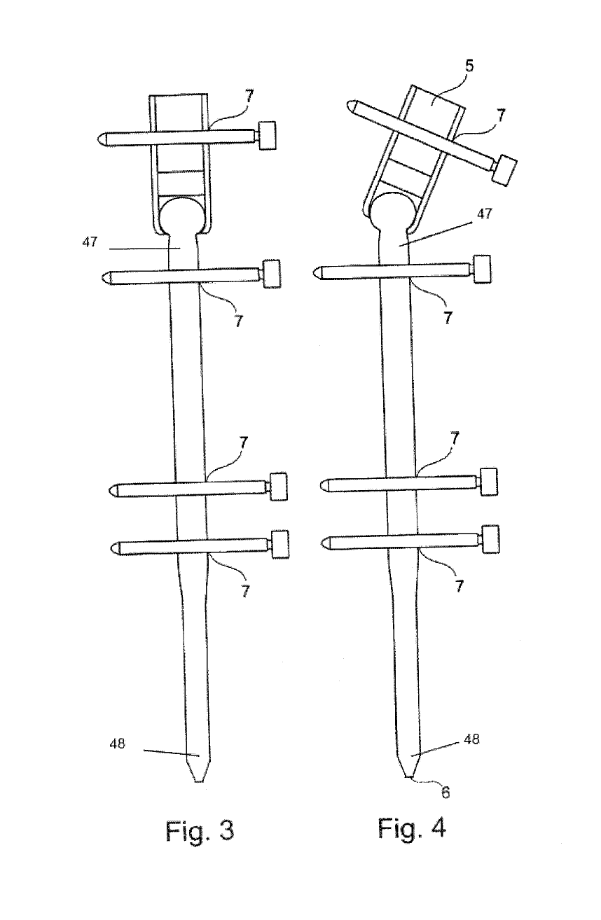

As shown in FIGS. 1-4, an intramedullary nail 1 according to an exemplary embodiment of the invention comprises a proximal portion 3 and a distal portion 2 connected to one another via a joint 4 such that the proximal and distal portions 3, 2 may rotate relative to one another, as shown in FIGS. 3 and 4, to adjust a final reduction of a fractured bone.

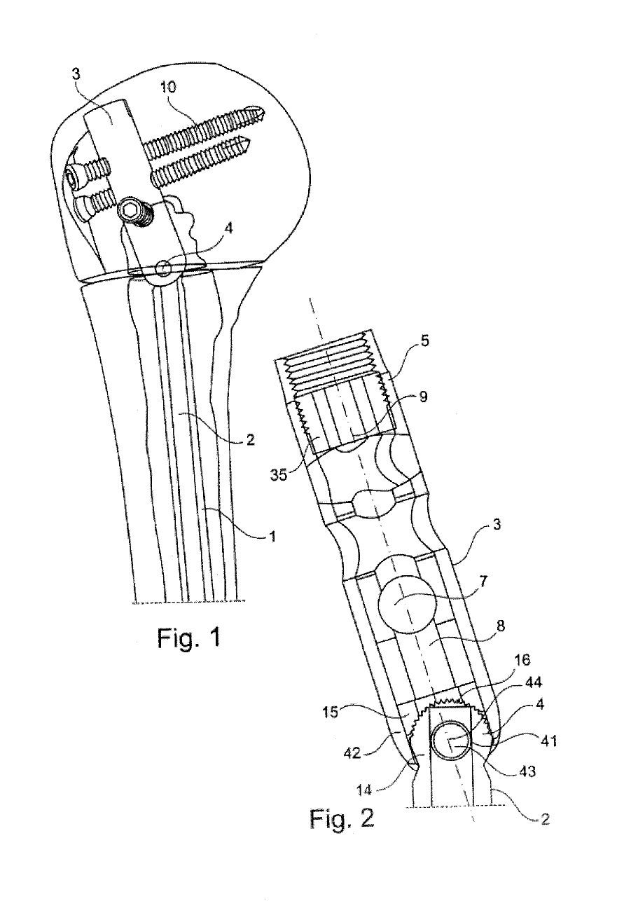

As shown in FIG. 2, the distal portion 2 extends longitudinally from a first end 47 connected to the proximal portion 3 to a second end 48 at a distal end of the nail 1. The distal portion 2 includes at least one locking hole 7 extending transversely therethrough. The locking hole 7 is sized and shaped to receive a locking screw 10 therethrough to fix the distal portion 2 of the nail 1 relative to the bone. The first end 47 includes a joint member 14 of the joint 4. The joint member 14 includes a partially spherical surface about a periphery thereof and opposing planar side surfaces. This configuration permits the joint member 14 to act as a hinge joint allowing rotation of the proximal and distal portions about one axis. The joint member 14 may include a plurality of teeth along the rounded portion of the outer surface. In one embodiment, the distal portion 2 may include a locking hole 7 proximate the joint member 14. The distal portion 2 may include additional locking holes 7 along a length thereof.

As shown in FIG. 2, the proximal portion 3 extends along a central axis 9 from a first end 5 at a proximal end of the nail 1 to a second end 42 connected to the first end 47 of the distal portion 2. The proximal end 3 includes a cavity 8 extending therethrough along the central axis 9 and at least one locking hole 7 extending transversely therethrough to receive a locking screw 10. In a preferred embodiment, the proximal portion 3 includes three locking holes 7. It will be understood by those of skill in the art, however, that the proximal portion 3 may include any number of locking holes 7. The proximal portion 3 further includes a transverse bore 41 extending therethrough proximate the second end 42 of the proximal part 3. The transverse bore 41 is sized and shaped to receive a bolt 43 inserted therethrough to couple the proximal and distal portions 3, 2 to one another. The transverse bore 41 has a bore axis 44 substantially orthogonal to the central axis 9 and which defines the axis of rotation of the proximal part 3 relative to the distal part 2. Where there are a plurality of locking holes 7 extending through the proximal part 3, the locking holes 7 may be axially spaced apart from one another. Further, the locking holes 7 may be arranged at different angles relative to a plane defined by the central axis 9 and the bore axis 44.

The cavity 8 receives an annular disc 15 therein at the second end 42. The disc 15 comprises a recess 16 complementarily shaped to the joint member 14. For example, the recess 16 may be partially spherical, including planar side surfaces corresponding to the outer surface of the joint member 14 such that the joint member 14 is received within the recess 16 at the second end 42 of the cavity 8. The spherical recess 16 may also include a plurality of teeth corresponding to the teeth arranged on the outer surface of the distal joint member 14 to permit the proximal and distal portions 3, 2 of the intramedullary nail 1 to be fixed at a desired angle relative to one another. The joint member 14 and the corresponding recess 16 of the joint 4 act as a hinge joint allowing only an angulation of the proximal part 3 relative to the distal part 2 in one plane. Angulations in two planes can be achieved by additionally rotating the intramedullary nail 1 inside the bone. Although the joint 4 is shown and described as a hinge joint, it will be understood by those of skill in the art that the joint 4 may be any joint that permits movement of the proximal portion 3 relative to the distal portion 2. For example, the joint 4 may also be a ball and socket joint or other similar joint mechanism.

The intramedullary nail 1 may also include a cylindrical sleeve 17 received therein proximally of the annular disc 15. At the proximal end 5 of the proximal part 3 the cavity 8 comprises an internal thread 18. Once the proximal and distal portions 3, 2 have been positioned at a desired angle relative to one another to properly reduce the fracture, a fastening screw 35 is inserted from the proximal end 5 into the cavity 8 engaging the internal thread 18. By tightening the fastening screw 35 the cylindrical sleeve 17 is pressed against the second end 42 of the proximal part 3 thereby pressing the disc 15 against the joint member 14 and locking the joint 4 at the desired angle. It will be understood by those of skill in the art that the intramedullary nail 1 may include more than one cylindrical sleeve 17 received therein.

The cylindrical sleeve 17 includes a transverse bore hole 19 located so that the transverse bore hole 19 receives a locking screw 10 therein when the locking screw 10 is inserted into the locking hole 7 in the proximal part 3. When the insert 35 is inserted into the first end 5 to lock the joint 4, once under pressure, the cylindrical sleeve 17 additionally locks the locking screws 10, preventing migration thereof. Specifically, the cylindrical sleeve 17 is moved distally relative to the proximal portion 3 such that an inner surface of the transverse bore hole 19 presses against a shaft portion of the locking screw 10 inserted therethrough. The above locking mechanism allows locking and unlocking of the joint 4 even after placement of the locking screws 10.

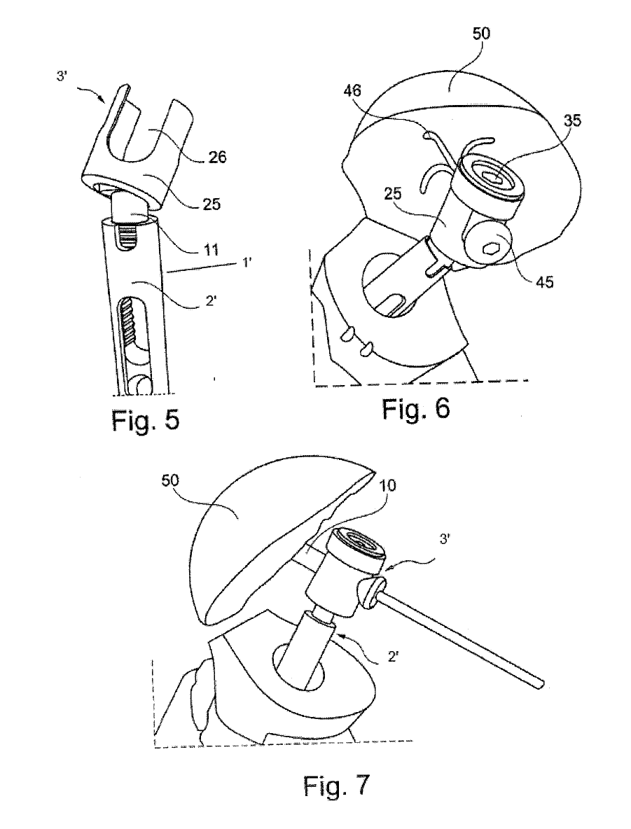

FIGS. 5 - 8 illustrate a further embodiment of an intramedullary nail 1′, which may be fixed to a bone using a rivet 45. The rivet 45 includes arms 46, which may spread radially outward to be fixed in the humeral head 50. Various embodiments of a bone anchor configured as a rivet are described in WO 99/62418 to FRIGG. The entire content of the aforementioned document is incorporated herein by reference thereto. The intramedullary nail 1′ is substantially similar to the intramedullary nail 1, as described above, comprising a proximal portion 3′ and a distal portion 2′ movable relative to one another via a joint 4′. The joint 4′ may include a connecting screw 11 including a spherical head 12 at a proximal end thereof for coupling to the proximal portion 3′ and an engaging element 49 at a distal end thereof for engaging the distal portion 2′. The spherical head 12 permits rotation of the proximal portion 3′ relative to the distal portion 2′. The engaging element 49 may include, for example, a thread along a portion thereof corresponding to a threaded inner surface at a proximal end 47′ of the distal portion 2′. Similarly to the intramedullary nail 1, the proximal and distal portions 3′, 2′ may be fixed at a desired angle relative to one another via a set screw 35′ inserted at a proximal end 27 of a sleeve 25 of the proximal portion 3′.

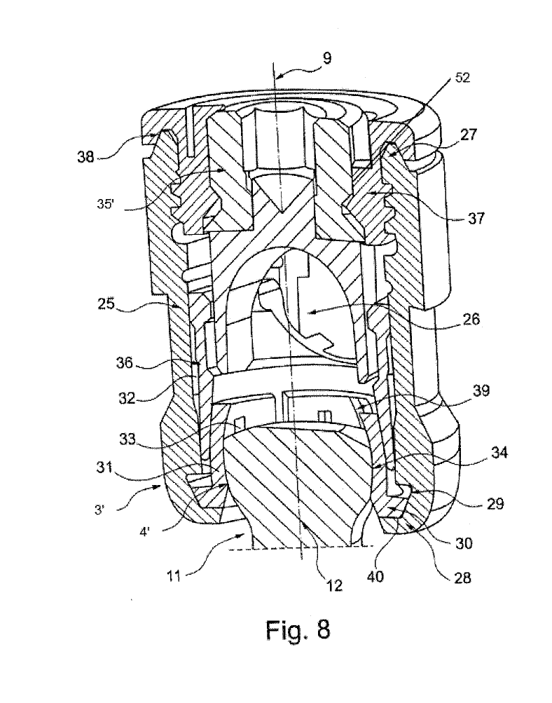

As illustrated in FIG. 8, the proximal portion 3′ includes a hollow cylindrical sleeve 25 and a radially compressible sleeve 31 arranged in the sleeve 25 and having a concave spherical cavity 34 forming a ball cup of the joint 4′ sized and shaped to receive the spherical head 12. The sleeve 25 is provided with a channel 26 open toward a proximal end 27 of the sleeve 25. The channel 26 also extends transversely through the sleeve 25 with an opening thereof sized and shaped to receive the rivet 45, as described above, a locking screw 10 or other bone anchoring element. The channel 26 may be formed, for example, as a substantially oval bore hole. The proximal portion 3′ may further include an insert screw 37 insertable into the proximal end 27 to provide an engagement structure for the set screw 35′ in the channel 26. A distal end 28 of the sleeve 25 may include a ring shaped recess 29 extending about an inner surface of the sleeve 25 to receive a portion of the radially compressible sleeve 31. When the radially compressible sleeve 31 is inserted in the bore hole 32 of the sleeve 25, a flange 30 of the radially compressible sleeve 310 is retained in the ring shaped recess 29. Thus, the radially compressible sleeve 31 is prevented from moving along a central axis 9 of the proximal portion 3′, while still permitting the radially compressible sleeve 31 to be radially movable.

The radially compressible sleeve 31 includes a concave spherical cavity 34 sized and shaped to receive the spherical head 12 of the joint 4′. The radially compressible sleeve 31 may include slots 33 extending alternately through an upper base area 39 and a lower base area 40 thereof to enable a homogeneous expansion and compression of the radially compressible sleeve 31. Due to the radial freedom of the radially compressible sleeve 31 in the bore hole 32 of the sleeve 25, the spherical head 12 of the connecting screw 11 may be snapped into and out of the radially compressible sleeve 31 as long as the proximal portion 3′ is not fixed relative to the distal portion 2′ via tightening of a set screw 35′.

The insert screw 37 may be inserted in and attached to the proximal end 27 of the sleeve 25 to receive the set screw 35′. Since the sleeve 25 is interrupted by the channel 26, the insert screw 37 may provide a continuous opening for engaging with the set screw 35′. Thus, the insert screw 37 includes an opening 52 extending longitudinally therethrough. The opening 52 includes an engagement structure (e.g., internal thread) extending along a surface thereof for engaging a corresponding engagement structure (e.g., an external thread) of the set screw 35′. The insert screw 37 may attach to the sleeve 25 via a groove 38 extending about a rim portion thereof, the groove 38 being sized and shaped to receive the proximal end 27 of the sleeve 25 therein. The groove 38 additionally prevents the proximal end 27 of the sleeve 25 from widening as the set screw 35′ is inserted and tightened therein.

On its external periphery, the radially compressible sleeve 31 tapers conically toward the upper base area 39 axially opposite the flange 30. A hollow cylindrical insert 36 having a lower end shaped complementarily conical to the cone at the radially compressible sleeve 31 slides within the bore hole 32 of the sleeve 25. The cylindrical insert 36 radially compresses the radially compressible sleeve 31 via the conical joining to fasten the spherical head 12 of the connecting screw 11 in the spherical cavity 34. This cone connection can be loosened via an inner shoulder at the upper end of the cylindrical insert 36, which is configured to abut a respective instrument. A locking screw 10 and the spherical head 12 of the connecting screw 11 can be simultaneously locked by fastening screw 35 which is screwed in coaxially into the insert screw 37 arranged at the proximal end 27 of the sleeve 25. The fastening screw 35 presses—while being tightened—onto the locking screw 10 which itself presses onto the cylindrical insert 36 and radially compresses the radially compressible sleeve 31 by pushing the cones arranged at the outside of the radially compressible sleeve 31 and at the inside of the insert 36 into one another. As the sleeve 31 is radially compressed, the sleeve 31 is locked relative to the spherical head 12 locking the proximal portion 3′ at a desired angle relative to the distal portion 2′.

Various embodiments of a device for connecting a longitudinal member, e.g. a locking screw with another longitudinal member, e.g. a pedicle screw including a polyaxially pivotable coupling between the device and a pedicle screw are described in WO 98/52482 SCHLAPFER. The entire content of the aforementioned document is incorporated herein by reference thereto.

Although the invention and its advantages have been described in detail, it should be understood that various changes, substitutions, and alterations may be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

What is claimed is:

1. An intramedullary nail extending from a proximal end to a distal end, comprising:
a distal portion extending longitudinally from the distal end to a first connecting end and including a first locking hole extending therethrough;
a proximal end extending longitudinally from a second connecting end to the proximal end of the nail and including a second locking hole extending transversely therethrough; and
a joint connecting the first and second connecting ends such that the distal and proximal portions are rotatable relative to one another about the joint.

2. The intramedullary nail according to claim 1, wherein the proximal portion includes a third locking hole extending transversely therethrough.

3. The intramedullary nail according to claim 1, wherein the distal portion includes a fourth locking hole extending transversely therethrough.

4. The intramedullary nail according to claim 1, wherein a ratio of a length of the distal portion L_{D} to a length of the proximal portion L_{P} is larger than 2.

5. The intramedullary nail according to claim 1, wherein the joint is located at a distance D≦L/3 from the proximal end, wherein Lisa total length of the nail.

6. The intramedullary nail according to claim 1, wherein one of the proximal portion and the distal portion do not include a threading therealong.

7. The intramedullary nail according to claim 1, wherein the joint is releasably lockable.

8. The intramedullary nail according to claim 7, further comprising a fastening screw engagable with the proximal end of the intramedullary nail to releasably lock the joint.

9. The intramedullary nail according to claim 7, wherein the joint includes joint members at the first and second connecting ends, the joint members including interlocking toothed surfaces engagable with one another to lock the distal and proximal portions relative to one another in a desired angulation relative to one another.

10. The intramedullary nail according to claim 7, wherein the joint includes a radially compressible sleeve comprising a concave spherical cavity sized and shaped to slidably receive a convex spherical male joint member.

11. The intramedullary nail according to claim 1, wherein the joint is a hinge joint permitting the distal and proximal portions to rotate relative to one another only about a single fixed rotational axis.

12. The intramedullary nail according to claim 1, wherein the intramedullary nail is sized and shaped to be inserted in the medullary canal of a long bone.

13. A system for treating a bone, comprising:
an intramedullary nail extending from a proximal end to a distal end and including:
a distal portion extending longitudinally from the distal end to a first connecting end and including a first locking hole extending therethrough;
a proximal portion extending longitudinally from a second connecting end to the proximal end of the nail and including a second locking hole extending transversely therethrough; and
a joint connecting the first and second connecting ends such that the distal and proximal portions are rotatable relative to one another about the joint; and
a fixation element insertable through one of the first and second locking elements.

14. The system according to claim 13, wherein the fixation element is a locking screw.

15. The system according to claim 13, wherein the fixation element is a rivet insertable into the second locking hole, the rivet including arms spreading radially outward to extend into a head portion of the bone.

16. The system according to claim 13, wherein the joint is releasably lockable to fix the proximal and distal portions in a desired angulation relative to one another.

17. The system according to claim 16, further comprising a fastening screw engagable with the proximal end of the intramedullary nail to releasably lock the joint.

18. The system according to claim 16, wherein the joint includes joint members at the first and second connecting ends, the joint members including interlocking toothed surfaces, which engage one another to lock the distal and proximal portions relative to one another.

19. The system according to claim 16, wherein the joint includes a radially compressible sleeve comprising a concave spherical cavity sized and shaped to slidably receive a convex spherical male joint member.

20. A method for treating a bone, comprising:
inserting an intramedullary nail into a medullary canal of a bone via an entry opening through a head portion of the bone, the intramedullary nail including proximal and distal portions connected to one another via a joint between connecting ends of the proximal and distal portions;
rotating the proximal and distal portions relative to one another about the joint to a desired angulation relative to one another to achieve a desired final reduction of a fracture of the bone; and
locking the proximal and distal portions at the desired angulation relative to one another.
